(19) United States
(12) Patent Application Publication
(10) Pub. No.: US 2025/0213875 A1
(43) Pub. Date: Jul. 3, 2025

(54) CONNECTOR ASSEMBLY FOR AN IMPLANTABLE MEDICAL DEVICE

(71) Applicant: Cardiac Pacemakers, Inc., St. Paul, MN (US)

(72) Inventors: Benjamin Haasl, Forest Lake, MN (US); James Michael English, Cahir (IE); Robert Allen Jones, Lake Elmo, MN (US); Bryan Johnson, Vadnais Heights, MN (US)

(21) Appl. No.: 18/990,971

(22) Filed: Dec. 20, 2024

Related U.S. Application Data
(60) Provisional application No. 63/616,196, filed on Dec. 29, 2023.

(51) Int. Cl. A61N 1/375

(52) U.S. Cl. CPC A61N 1/3752

(57) ABSTRACT

A connector port assembly for releasably securing an implantable lead to a header of an implantable medical device comprises a connector block having a connector block bore and an access hole extending radially through the connector block and oriented orthogonally to an axis of the connector block bore and sized to receive a shaft of a release tool, and a collet assembly including a collet body and a plurality of bearings arranged about a circumference of the collet body, the collet body including a forward end portion having a curved outer surface, and a distal portion opposite the forward end portion, wherein the access hole of the connector block is positioned such that a shaft of the release tool can engage the curved outer surface of the forward end portion of the collet body.

### CROSS REFERENCE TO RELATED APPLICATIONS

The present applications claims priority to, and the benefit of, U.S. Provisional Patent Application No. 63/616,196, filed Dec. 29, 2023, the entire disclosure of which is incorporated herein by reference.

### TECHNICAL FIELD

The present disclosure relates to an implantable system having an implantable lead and a connector port. More specifically, the disclosure relates to releasably securing of the implantable lead within the connector port.

### BACKGROUND

Implantable medical systems may include an implantable lead assembly and an implantable pulse generator connected with the implantable lead assembly. Further, a header of the implantable pulse generator generally includes corresponding connector ports to effectively couple the lead assembly with the implantable pulse generator. A proper connection between the implantable leads and the corresponding connector ports is required to allow proper functioning of the implantable system. The lead assembly and the implantable pulse generator are to remain connected after implantation to ensure desired functionality.

### SUMMARY

In Example 1, an implantable medical device comprising a housing, a header arranged with the housing, and a connector port assembly arranged within the header and configured to couple an implantable lead to the header. The connector port assembly includes a connector block and a collet assembly. The connector block has a proximal end, an opposite distal end, a connector block bore extending between the proximal and distal ends, and an access hole extending radially through the connector block between the proximal and distal ends of the connector block. The collet assembly is arranged within the connector block bore and is configured to clamp against a portion of the implantable lead and to secure the implantable lead with the header in response to insertion of the portion of the implantable lead into the connector block bore, wherein the collet assembly includes a collet body and a plurality of bearings. The collet body has a proximal portion including a forward end portion oriented toward the proximal end of the connector block, and a distal portion oriented toward the distal end of the connector block, wherein the forward end portion has a curved outer surface. The plurality of bearings are arranged about the collet body between the distal end portion and the forward end portion of the collet body and configured to contact an inner circumference of the connector block. The access hole is aligned with the forward end portion of the collet body and is configured to receive a release tool for engaging the curved outer surface of the forward end portion of the collet body to cause the collet body to move in an axial direction toward the distal end of the connector block.

In Example 2, the implantable medical device of Example 1, wherein the connector block further comprises a release tool retaining feature configured to frictionally retain the release tool within the access hole in engagement with the curved outer surface of the forward end portion of the collet body.

In Example 3, the implantable medical device of Example 2, wherein the release tool retaining feature is a resilient member extending across the access hole.

In Example 4, the implantable medical device of Example 3, wherein the resilient member includes opposite end portions that are each retained within a slot formed in the connector block adjacent to the access hole, and a center portion that spans across the access hole.

In Example 5, the implantable medical device of any of Examples 1-4, wherein the plurality of bearings translate toward the distal end of the connector block in response to insertion of the portion of the implantable lead into the connector block bore.

In Example 6, the implantable medical device of any of Examples 1-5, further comprising a spring element disposed within the connector block bore and about the distal end portion of the collet body, the spring element configured to bias the collet body toward the proximal end of the connector block.

In Example 7, the implantable medical device of Example 6, wherein the spring element has a serpentine shape when viewed in a direction orthogonal to a longitudinal axis of the connector port.

In Example 8, the implantable medical device of any of Examples 1-7, wherein the distal portion of the collet body is dimensioned to maintain axial alignment of the collet body during axial translation of the collet body.

In Example 9, the implantable medical device of any of Examples 1-8, wherein an intermediate portion of the proximal portion of the collet body has an angled outer circumference and an inner surface of the connector block bore includes an angled inner circumference.

In Example 10, the implantable medical device of Example 9 wherein an angle of the angled outer circumference of intermediate portion of the proximal portion of the collet body is approximately equal to an angle of the angled inner circumference of the connector block bore.

In Example 11, the implantable medical device of Example 10, wherein both a cross section of the angled outer circumference of the intermediate portion of the proximal portion of the collet body and a cross section of the angled inner circumference of the connector block bore are tapered in a direction from a distal end of the collet body to a proximal end of the collet body, and wherein the angle is between approximately 0.5 degrees and approximately 35 degrees.

In Example 12, a medical system comprising the implantable medical device of any of Examples 1-11; and a release tool including a handle portion, and a release tool shaft extending from the handle portion and dimensioned to be inserted into the access hole of the connector block.

In Example 13, the medical system of Example 12, wherein the release tool shaft has a proximal portion extending from the handle portion, and a distal portion with a distal end, wherein the release tool shaft is dimensioned such that the distal end of the release tool shaft engages the curved outer surface of the forward end portion of the collet body when the release tool shaft is fully inserted into the access hole.

In Example 14, the medical system of Example 13, wherein the distal end of the release tool shaft has a chamfered or curved profile.

In Example 15, the medical system of Example 13, wherein the release tool shaft includes a circumferential notch positioned so form a releasable snap-fit with the release tool retaining feature.

In Example 16, an implantable medical device comprising a housing. a header arranged with the housing; and a connector port assembly arranged within the header and configured to couple an implantable lead to the header, the connector port assembly including a connector block and a collet assembly. The connector block has a proximal end, an opposite distal end, a connector block bore extending between the proximal and distal ends, and an access hole extending radially through the connector block between the proximal and distal ends of the connector block, the access hole oriented generally orthogonally to an axis of the connector block bore. T collet assembly includes a collet body and a plurality of bearings arranged about a circumference of the collet body, the plurality of bearings configured to contact an inner surface of the connector block bore and to frictionally engage a portion of the implantable lead to secure the implantable lead with the header in response to insertion of the portion of the implantable lead into the connector block bore, the collet body including a forward end portion having a curved outer surface, wherein the access hole of the connector block is aligned with curved outer surface of the forward end portion of the collet body.

In Example 17, the implantable medical device of Example 16, wherein the plurality of bearings translate toward the distal end of the connector block in response to insertion of the portion of the implantable lead into the connector block bore.

In Example 18, the implantable medical device of Example 17, further comprising a spring element disposed within the connector block bore and about the distal end portion of the collet body, the spring element configured to bias the collet body toward the proximal end of the connector port.

In Example 19, the implantable medical device of Example 18, wherein the spring element has a serpentine shape when viewed in a direction orthogonal to a longitudinal axis of the connector port.

In Example 20, the implantable medical device of Example 19, wherein the distal portion of the collet body is dimensioned to maintain axial alignment of the collet body during axial translation of the collet body.

In Example 21, an implantable medical device comprising a housing, a header arranged with the housing. a connector block and a collet assembly. The connector block is arranged within the header and has a proximal end, an opposite distal end, a connector block bore extending between the proximal and distal ends, and an access hole extending radially through the connector block between the proximal and distal ends of the connector block, the access hole being oriented orthogonally to an axis of the connector block bore and sized to receive a shaft of a release tool, the connector block further including a release tool retaining feature. The collet assembly includes a collet body and a plurality of bearings arranged about a circumference of the collet body, the plurality of bearings configured to contact an inner surface of the connector block bore and to frictionally engage a portion of an implantable lead to secure the implantable lead with the header in response to insertion of the portion of the implantable lead into the connector block bore, the collet body including a forward end portion and a distal portion opposite the forward end portion. The access hole of the connector port is positioned such that a shaft of the release tool can engage the forward end portion of the collet body, and the release tool retaining feature is configured to frictionally retain the release tool within the access hole in engagement with the forward end portion of the collet body.

In Example 22, the implantable medical device of Example 21, wherein the tool retaining feature is a resilient member extending across the access hole.

In Example 23, the implantable medical device of Example 22, wherein the resilient member includes opposite end portions that are each retained within a slot formed in the connector block adjacent to the access hole, and a center portion that spans across the access hole.

In Example 24, the implantable medical device of Example 23, wherein the plurality of bearings translate toward the distal end of the connector block in response to insertion of the portion of the implantable lead into the connector block bore.

In Example 25, the implantable medical device of Example 24, further comprising a spring element disposed within the connector block bore and about the distal end portion of the collet body, the spring element configured to bias the collet body toward the proximal end of the connector block.

In Example 26, the implantable medical device of Example 25, wherein the spring element has a serpentine shape when viewed in a direction orthogonal to a longitudinal axis of the connector port.

In Example 27, the implantable medical device of Example 26, wherein the distal portion of the collet body is dimensioned to maintain axial alignment of the collet body during axial translation of the collet body.

In Example 28, a connector port assembly for releasably securing an implantable lead to a header of an implantable medical device, the connector port assembly comprising a connector block and a collet assembly. The connector block has a proximal end, an opposite distal end, a connector block bore extending between the proximal and distal ends, and an access hole extending radially through the connector block between the proximal and distal ends of the connector block, the access hole being oriented orthogonally to an axis of the connector block bore and sized to receive a shaft of a release tool. The collet assembly includes a collet body and a plurality of bearings arranged about a circumference of the collet body, the plurality of bearings configured to contact an inner surface of the connector block bore and to frictionally engage a portion of an implantable lead to secure the implantable lead with the connector port assembly in response to insertion of the portion of the implantable lead into the connector block bore, the collet body including a forward end portion having a curved outer surface, and a distal portion opposite the forward end portion. The access hole of the connector block is positioned such that a shaft of the release tool can engage the curved outer surface of the forward end portion of the collet body.

In Example 29, the connector port assembly of Example 28, wherein the connector block further comprises a release tool retaining feature configured to frictionally retain the release tool within the access hole in engagement with the curved outer surface of the forward end portion of the collet body.

In Example 30, the connector port assembly of Example 29, wherein the release tool retaining feature is a resilient member extending across the access hole.

In Example 31, the connector port assembly of Example 30, wherein the resilient member includes opposite end portions that are each retained within a slot formed in the connector block adjacent to the access hole, and a center portion that spans across the access hole.

In Example 32, the connector port assembly of Example 31, wherein the plurality of bearings translate toward the distal end of the connector block in response to insertion of the portion of the implantable lead into the connector block bore.

In Example 33, the connector port assembly of Example 29, further comprising a spring element disposed within the connector block bore and about the distal end portion of the collet body, the spring element configured to bias the collet body toward the proximal end of the connector block.

In Example 34 the implantable medical device of Example 33, wherein the spring element has a serpentine shape when viewed in a direction orthogonal to a longitudinal axis of the connector port.

In Example 35, the connector port assembly of Example 29, wherein the distal portion of the collet body is dimensioned to maintain axial alignment of the collet body during axial translation of the collet body.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an illustration of an example implantable system for stimulating a target location on or within the heart, in accordance with various aspects of the present disclosure.

FIG. 2A is a perspective view of an illustration of an example connector port assembly, in accordance with various aspects of the present disclosure.

FIG. 2B is an exploded perspective view of the connector port assembly of FIG. 2A, in accordance with various aspects of the present disclosure.

FIG. 2C is an elevation view of a component of the connector port assembly of FIG. 2A, in accordance with various aspects of the present disclosure.

FIG. 2D is a top view of the connector port assembly of FIG. 2A, in accordance with various aspects of the present disclosure.

FIG. 2E is a cross-sectional illustration of the connector port assembly of FIG. 2A taken along the line 2D-2D in FIG. 2C, in accordance with various aspects of the present disclosure.

FIG. 2F is a perspective illustration of a spring element configured for use in the connector port assembly of FIG. 2A, in accordance with various aspects of the present disclosure.

FIG. 3 is an elevation view of an exemplary release tool for use with the connector port assembly of FIGS. 2A-2E, in accordance with various aspects of the present disclosure.

FIGS. 4A and 4B illustrate the release tool of FIG. 3 in use in conjunction with the connector port assembly of FIGS. 2A-2E, in accordance with embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

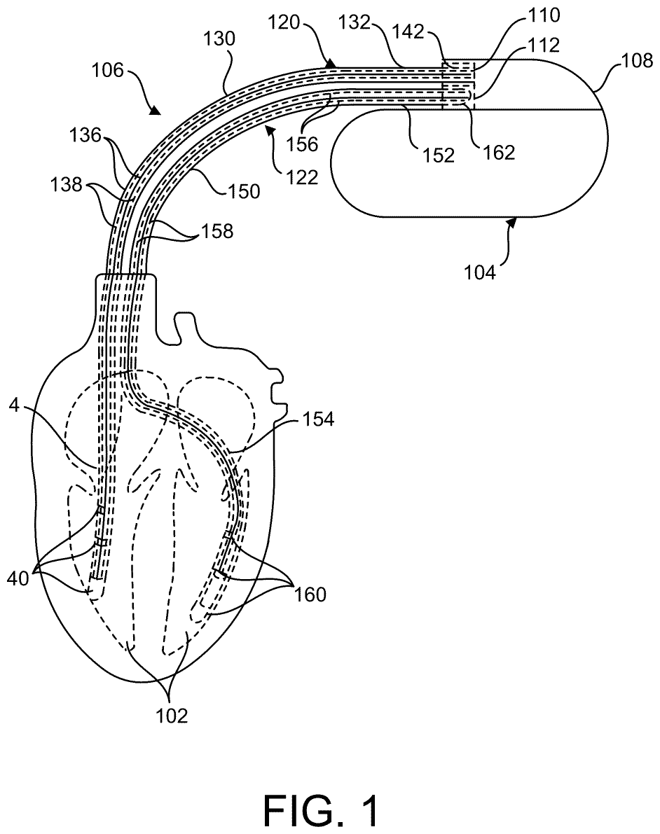

FIG. 1 is a schematic illustration of an implantable system 100 for stimulating a target location 102 on or within the heart. As shown, the implantable system 100 includes an implantable medical device (IMD) 104 and an implantable lead assembly 106 connected to the IMD 104. In various embodiments, the IMD 104 is an implantable pulse generator adapted to generate electrical signals to be delivered to the target location 102 for pacing and/or for sensing electrical activity at a location on or within the heart. The IMD 104 can include microprocessors to provide processing, evaluation, and to deliver electrical shocks and pulses of different energy levels and timing for defibrillation, cardioversion, and pacing to a heart in response to cardiac arrhythmia including fibrillation, tachycardia, heart failure, and bradycardia. In other instances, the implantable system 100 can also be suitable for use with implantable electrical stimulators, such as, but not limited to, neuro-stimulators, skeletal stimulators, central nervous system stimulators, or stimulators for the treatment of pain.

The IMD 104 may include one or more connector ports 110, 112. In certain instances, the IMD (e.g., pulse generator 104) includes a header 108 with the connector port(s) 110, 112. As shown, for example, the header 108 includes a first connector port 110 and a second connector port 112. In addition, the implantable lead assembly 106 includes a first implantable lead 120 connected to the first connector port 110 and a second implantable lead 122 connected to the second connector port 112. In some instances, the implantable lead assembly 106 may also include a third implantable lead (not shown) and the header 108 may include a corresponding third connector port (not shown).

Each of the first and second implantable leads 120, 122 includes a flexible lead body, a plurality of conductor wires, a plurality of electrodes, and a terminal connector assembly (as shown in detail, for example, with reference to FIGS. 5-6). For example, as shown, the first implantable lead 120 includes a flexible lead body 130 having a proximal end 132, a distal end portion 134, and a plurality of conductor lumens 136 extending axially within the lead body 130 from the proximal end 132 to the distal end portion 134. The first implantable lead 120 also includes a plurality of conductor wires 138, each conductor wire extending within one of the conductor lumens 136 in the lead body 130. The first implantable lead 120 further includes a plurality of electrodes 140 coupled to the distal end portion 134 of the lead body 130. Each of the electrodes 140 are electrically coupled to at least one of the plurality of conductor wires 138. The first implantable lead 120 also includes a terminal connector assembly 142 (or terminal pin) coupled to the proximal end 132 of the lead body 130. The terminal connector assembly 142 is sized to be inserted into and received by the first connector port 110 of the header 108.

Similarly, the second implantable lead 122 includes a flexible lead body 150 having a proximal end 152, a distal end portion 154, and a plurality of conductor lumens 156 extending axially within the lead body 150 from the proximal end 152 to the distal end portion 154. The second implantable lead 122 also includes a plurality of conductor wires 158, each conductor wire extending within one of the conductor lumens 156 in the lead body 150. Further, the second implantable lead 122 includes a plurality of electrodes 160 coupled to the distal end portion 154 of the lead body 150. Each of the electrodes 160 are electrically coupled to at least one of the plurality of conductor wires 158. The second implantable lead 122 also includes a terminal connector assembly 162 coupled to the proximal end 152 of the lead body 150. The terminal connector assembly 162 is sized to be inserted into and received by the second connector port 112 of the header 108.

As an example of implant locations for one or more leads, the first implantable lead 120 is shown extending into a right ventricle of the heart, and the second implantable lead 122 extending through the coronary sinus and into a coronary vein disposed outside the left ventricle of the heart. The electrical signals and stimuli conveyed by the IMD 104 are carried to the electrode at the distal end of the lead by the conductors. The IMD 104 is typically implanted subcutaneously within an implantation location or pocket in the patient's chest or abdomen.

The IMD 104 and lead(s) 120, 122 are connected by a physician. In order to maintain the implantable leads 120, 122 connected to the IMD 104, the header 108 may include a mechanism (e.g., a collet assembly) that secures the lead(s) 120, 122 in place. As used herein, a collet assembly can refer to a number of subsystems such as a ball, roller, and/or cam with a basing member. A number of balls, rollers, and/or cams can be captured in a collet and a biasing member may engage the collet to ensure location accuracy of the collet assembly. In a first example, the collet assembly can generally act as a container for the balls, rollers, and/or cams. In a second example, the collet assembly can be one or more collets and can have a friction bias between the implantable leads 120, 122 and the one or more collets. In this way, a higher friction exists between the implantable leads 120, 122 and the one or more collets than between the housing and the one or more collets. This friction bias can be achieved by one or more methods, including but not limited to surface topology optimization, coatings, or having a roller, ball, and/or cam between the collet and housing. As used hereinafter, collet assembly may refer to any of the aforementioned examples, including the first and second examples. In addition, the securement mechanism may be releasable, in certain instances, to allow for manipulation or replacement of the lead(s) 120, 122. The securement mechanism may lessen the ability for the lead(s) 120, 122 to be removed or back out of the header 108, without intentional intervention by the physician, after the lead(s) 120, 122 have been connected. Further yet, the securement mechanism does not require any additional tool or tooling (e.g., a set screw) for connection of the lead(s) 120, 122 to the header 108.

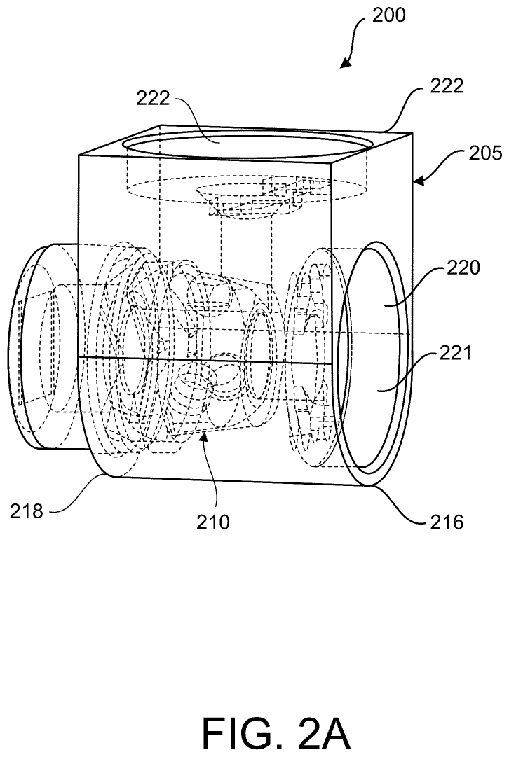

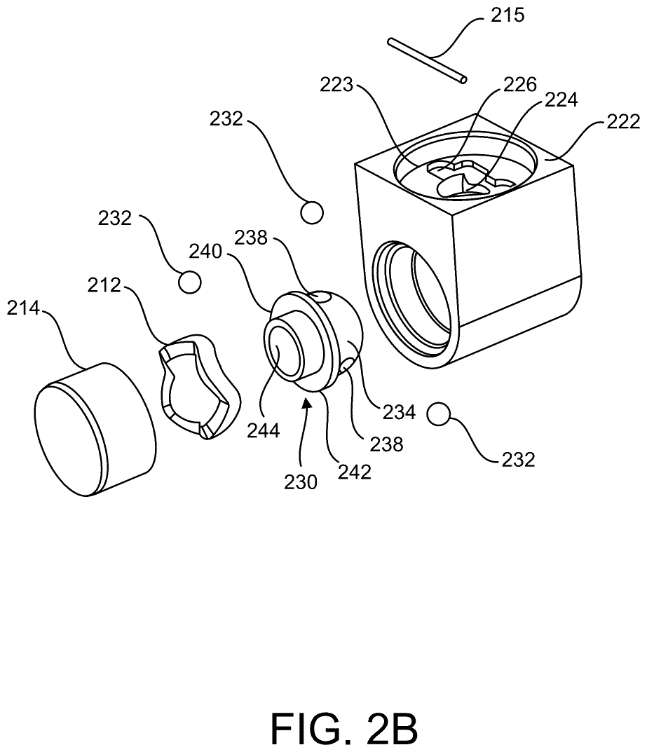

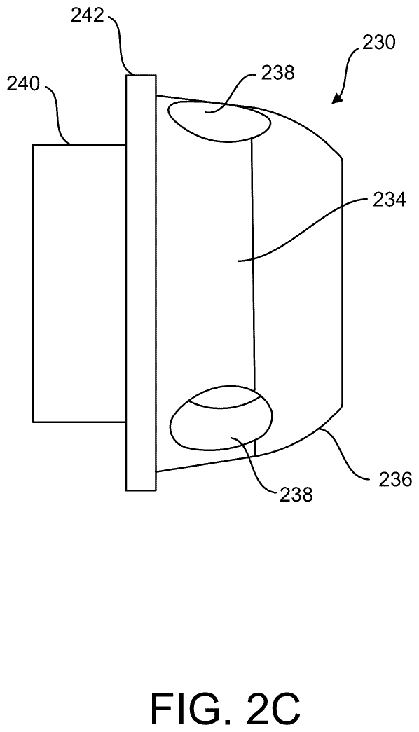

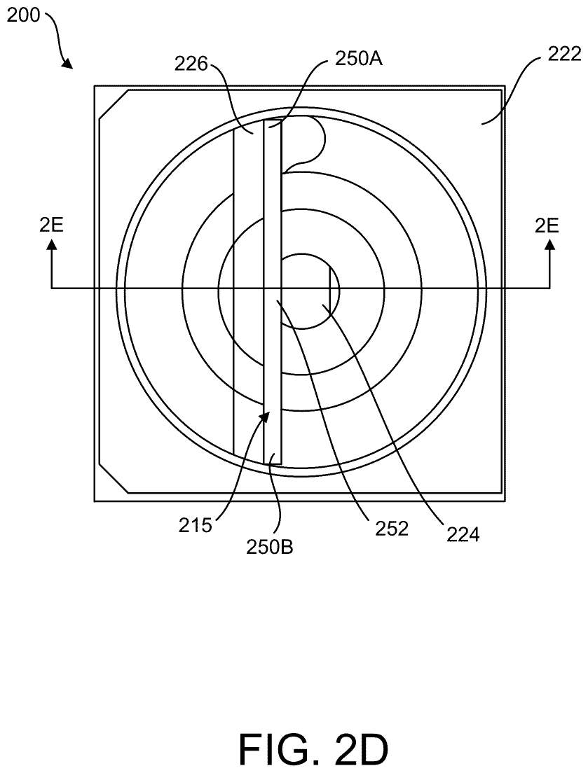

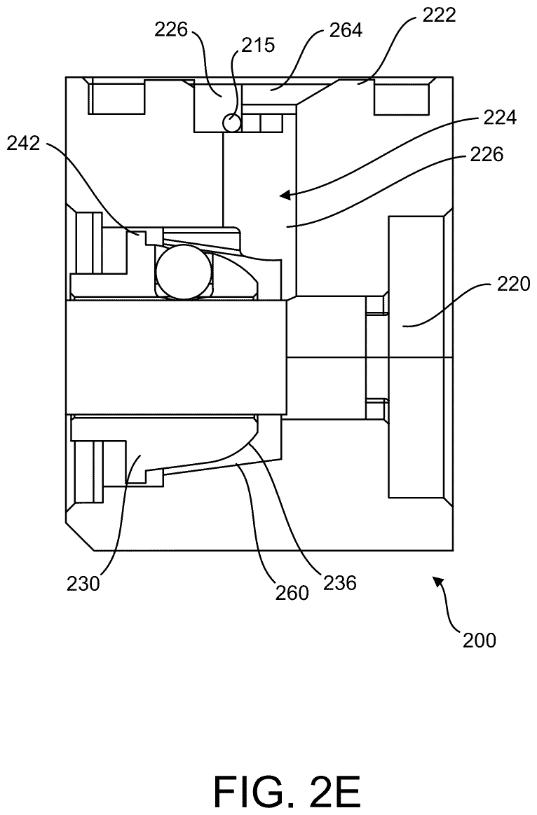

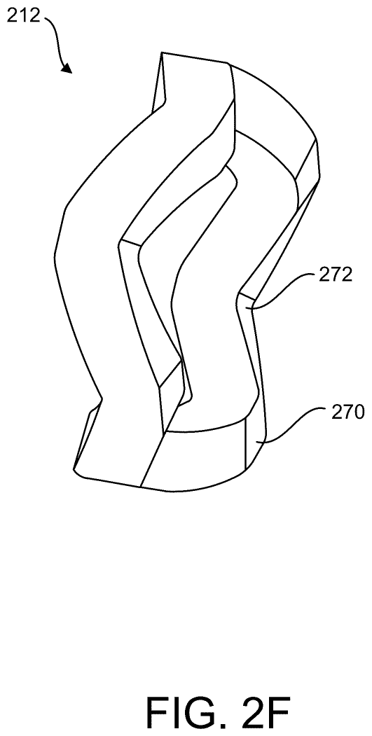

FIGS. 2A and 2B are perspective and exploded views, respectively, of an example connector port assembly 200 in accordance with various aspects of the present disclosure. The connector port assembly 200 may be arranged within a header that forms a portion of a housing of an implantable medical device as shown above with reference to FIG. 1. The connector port assembly 200, arranged within the header, is configured to couple an implantable lead to the header (and to the implantable medical device).

As shown, the connector port assembly 200 includes a connector block 205, a collet assembly 210, a spring element 212, an end cap 214 and a release tool retainer 215. In the illustrated embodiment, the connector block 205, which may be a machined or molded component, has a proximal end 216, a distal end 218, and a connector block bore 220 extending axially from a proximal opening 221 at the proximal end 216 through the distal end 218. As further shown, the connector block 205 has an upper surface 222 with a recess 223 extending inwardly (i.e., toward the connector block bore 220), and an access hole 224 located within the recess 223. In the illustrated embodiment, a tool retainer slot 226 is formed in a lower face of the recess 223. Referring in particular to FIG. 2B, the collet assembly 210 has a collet body 230 and a plurality of bearings 232.

FIG. 2C is an elevation view of the collet body 230. As shown, the collet body 230 has a proximal portion 234 having a forward end portion 236 defining an outer surface with curved/contoured profile, and a plurality of openings 238 disposed equidistantly about a circumference of the proximal portion 234. The collet body 230 further has a distal portion 240 having an outer diameter that is smaller than the maximum diameter of the proximal portion 234, and a flange 242 is disposed between the proximal and distal portions 234, 240. As further shown in FIG. 2B, the collet body 230 has a central collet bore 244.

Referring to FIGS. 2A-2C collectively, in embodiments, the collet assembly 210 is slidably disposed within the connector block bore 244, with the collet bore 244 located coaxially with the connector block bore 220, such that the collet bore 244 can receive a terminal pin of an implantable lead connector inserted through the proximal opening 220 of the connector block 205 (see FIG. 1). Additionally, each of the bearings 232 is disposed within a respective one of the openings 238 in the collet body 230, with a portion of each bearing 232 extending into the collet bore 244 and another portion extending radially outward of the outer surface of the collet body 244. In this configuration, each bearing 232 is configured to contact and engage the terminal pin of the implantable lead inserted into the collet bore 244 as well as an inner surface of the connector block bore 220 to releasably secure the terminal pin to the connector port assembly 200, as will be explained in further detail herein. Additionally, the spring element 212 is disposed about an outer surface of the distal portion 240 of the collet body 230 and is axially constrained between the flange 242 and a surface of the end cap 214 such that axial translation of the collet assembly 230 relative to the connector block 205 and the end cap 214 compresses the spring element 212, which in turn creates an axial force tending to resist such axial movement of the collet assembly 210, thereby biasing the collet assembly 210 toward the proximal end 216 of the connector block 205.

FIG. 2D is a top view of the connector port assembly 200, and FIG. 2D is a cross-sectional illustration of the connector port assembly 200 taken along the line 2E-2E in FIG. 2D, in accordance with various aspects of the present disclosure. As shown, the release tool retainer 215 has opposite end portions 250A, 250B, and a center portion 252. The release tool retainer 215 is disposed within tool retainer slot 226, with the end portions 250A, 250B abutting and constrained by a wall circumscribing the recess 223 in the upper surface 222 of the connector block 250. As shown, the center portion 252 of the release tool retainer 215 is disposed over and spans the access hole 224. With reference to FIG. 2E, the connector block 205 has an inner circumferential surface 260 defining the connector block bore 220. As shown, the inner circumferential surface 260 is angled when viewed in cross-section, which facilitates engagement of the medical lead terminal pin in use, as explained in greater detail below.

The access hole 224 has an upper portion 264 originating at the upper surface 222 of the connector block 205, and a lower portion 266 extending inwardly to the connector block bore. The upper portion 264 has a generally conical profile when viewed in cross section, and the lower portion 266 extends from the upper portion 264 and has a lateral dimension that is sized to receive a shaft of a release tool, as explained in greater detail below. As further shown, particularly in FIG. 2E, the forward end portion 236 of the collet body 230 extends partially into the lower portion 266 of the access hole 224 and is positioned so as to be engageable by the release tool shaft to facilitate release of the terminal pin of the medical lead from the connector port assembly 200, as will further be explained in greater detail below. The upper portion 264 of the access hole 224 has a surface operable to interact with a feature on the shaft of the release tool to delimit the depth of insertion of the release tool into the access hole.

In the illustrated embodiment, the release tool retainer 215 is in the form of a rod or pin constructed of a sufficiently flexible and resilient material (which can be a metal or polymeric material) so as to allow the center portion 252 to bend or deflect laterally (i.e., away from the center of the access hole) upon insertion of the release tool shaft into the access hole 224. The release tool retainer 215 is thus operable to frictionally engage a release tool shaft when inserted into the access hole 224 so as to retain the release tool shaft within the access hole 224 during removal of the terminal pin of the medical lead from the connector port assembly 200.

FIG. 2F is an isometric illustration of the spring element 212, in accordance with embodiments of the present disclosure. As shown, the spring element 212 has a generally serpentine configuration formed of alternating peaks 270 and valleys 272 circumscribing the spring element 212. In embodiments, the spring element 212 is constructed of a material (polymeric, elastomeric, or metallic) selected to impart sufficient resiliency to the spring element 212 such that upon axial compression (e.g., between the end cap 214 and the flange 242 of the collet body 230) during axial translation of the collet assembly 210 creates a resultant axial spring force tending to bias the collet assembly 210 toward the proximal end 216 of the connector block). In one embodiment, the spring element 212 is formed of a silicone material. As will be appreciated by the skilled artisan based on this present disclosure, the foregoing axial force results from flattening of the peaks 270 and valleys 272 due to axial compression of the spring element 212. The inventors of the present disclosure have discovered that the serpentine configuration of the spring element 212 tends to provide a biasing force on the collet assembly 210 that is more uniformly distributed about its circumference than, for example, a helical spring. This in turn promotes maintenance of axial alignment of the collet body 230 within the connector block bore 205 (i.e., by inhibiting rotation of the collet body 230 about an axis orthogonal to the axis of the connector block bore which could tend to cause binding of the collet assembly 210 during axial translation). In some embodiments, however, a different spring configuration may be utilized within the scope of the present disclosure.

Referring collectively to FIGS. 2A-2F, the inner circumferential surface 260 of the connector block 205 and an intermediate portion of the proximal portion 234 of the collet body 230 are angled outward in the proximal-to-distal direction. Additionally, the bearings 232 are positioned to contact the inner circumferential surface 260 of the connector block 205. In certain instances, the one or more bearings 232 may include three or more bearings 232, although in other embodiment, a different number of bearings 232 may be employed. The position of the bearings 232 operates to centrally or symmetrically align the collet assembly 210 within the connector block 205. The bearings 232 may take a variety of forms, such as a bearing ball or a rolling needle. Any number (e.g., one, two, three, etc.) or size of bearings 232 discussed herein should not be interpreted to limit the number of bearings 232 that may be included in the present disclosure. There may be an odd number of bearings 232 in some instances while in other instances there may be an even number of bearings 232. The bearings 232 may facilitate movement of the collet assembly 210 within the connector block 205. The collet assembly 210 may only be held in position or constrained in the direction opposite to a direction of insertion of the implantable lead. Under these circumstances, the collet assembly 210 may otherwise be free to rotate, axially translate, or radially translate within the connector assembly, within tolerances.

In any of these examples, the bearings 232 may allow the collet assembly 210 to be movable within the connector block 205 such that the one or more bearings 232 may translate toward or away from the distal end 218 of the connector block 205 in response to insertion of a portion of an implantable lead within the connector port assembly 200.

A portion (e.g., a terminal pin) of the implantable lead may be inserted into the connector port assembly 200 at the proximal opening 221 of the connector block 205 therein by movement in a direction from the proximal end 216 toward the distal end 218 of the connector block 205. Under these circumstances, a portion of the implantable lead that is inserted into the connector port assembly 200 may be received and frictionally engaged by the collet assembly 210. During insertion, the one or more bearings 232 may translate along the implantable lead as the implantable lead is progressively received within the collet assembly 210. As such, the portion of the implantable lead may engage the one or more bearings 232 of the collet assembly 210. When engaged, the one or more bearings 232 may cause the collet assembly 210 to inhibit movement of the portion of the implantable lead in one or more directions relative to the collet assembly 210. The one or more bearings 232 may inhibit axial movement of the implantable lead within the collet assembly 210 in at least one direction such that the portion of the implantable lead is secured within the connector port assembly 200. The one or more bearings 232 of the collet assembly 210 may be configured to maintain the portion of the implantable lead within the connector port assembly 200 in response to forces acting on the implantable lead in a direction generally from the distal end 218 toward the proximal end 216 of the connector block. After insertion, the implantable lead is removably secured within the connector port assembly 200.

In certain instances, the one or more bearings 232 may frictionally engage the portion of the implantable lead after insertion into the connector port assembly 200 and the angled inner circumferential surface 260 of the connector block 205. When the one or more bearings 232 frictionally engage the portion of the implantable lead, the one or more bearings 232 may cause the collet assembly 210 to move in the same direction (the direction of insertion of the portion of the implantable lead into the connector port assembly 200). The one or more bearings 232 reduce the friction between the implantable lead and the collet while maintaining contact with the portion of implantable lead. During insertion, the implantable lead may move further distally than the collet assembly 210. In certain instances, the one or more bearings 232 may translate toward the distal end 218 of the connector block 205 in response to insertion of the portion of the implantable lead into the connector block 205. After insertion, the one or more bearings 232 may translate away from the distal end 218 of the connector block 205. In response to insertion, the angled inner circumferential surface 260 of the connector block 205 can facilitate movement of the collet assembly 210 toward the proximal end 216 of the connector block 205 as the one or more bearings 260 engage the angled inner circumferential surface 260 thereby exerting compressive or radially inward forces on the portion the implantable lead arranged within the collet assembly 210.

Arrangement of the one or more bearings 232 about the collet assembly 210 may take numerous forms. For example, arrangement of the one or more bearings 232 may be varied circumferentially, axially, and radially. In certain instances, the one or more bearings 232 may include a plurality of bearings 232. The plurality of bearings 232 may be spaced equally about the circumference of the collet assembly 210. In another instance, the plurality of bearings 232 may be spaced variedly about the circumference of the collet assembly 210. With regards to axial arrangements, each bearing in the plurality of bearings 232 may be arranged in plane with each other, for example, at a plane extending through a circumference of the collet assembly 210. In other instances, a number (e.g., one, two, three, etc.) of bearings 232 in the plurality of bearings 232 may be out of plane with the other (e.g., one, two, three, etc.) bearings 232 in the plurality of bearings 232. With regards to radial arrangements, similarly, each bearing in the plurality of bearings 232 may be equally radially spaced from a central axis of the collet assembly 210 or, on the other hand, may be variedly radially spaced from a central axis of the collet assembly 210. In any of these examples, the one or more bearings 232 may be arranged such that they do not detrimentally contact certain features of the connector port assembly 200 (e.g., the access hole 224 for removing the implantable leads as discussed further hereinafter).

As noted above and in certain instances, the one or more bearings 232 may be forced against the portion of the implantable lead and a portion of the inner circumferential surface 260 of the connector block 205. In this way, the one or more bearings 232 may forcibly engage or collapse toward and engage the portion of the implantable lead. In certain instances, the one or more bearings 232 may be configured to contact the portion of the implantable lead and the inner circumferential surface 260 of the connector block 205 with substantially equal forces. In certain instances, the one or more bearings 232 may maintain compressive forces acting on the portion of the implantable lead in response to the forces acting on the implantable lead in a direction generally from the distal end 218 toward the proximal end 216 of the connector block 205. Under these circumstances, a magnitude of the compressive forces may be proportional to a magnitude of the forces acting on the implantable lead in a direction generally from the distal end 218 toward the proximal end 216.

In certain instances, the collet assembly 210 may be biased toward one end of the connector port assembly 200. The collet assembly 210 may be biased toward the proximal end 216 of the connector block 205. In this manner, a location of the collet assembly 210, prior to the portion of the implantable lead being inserted into the collet assembly 210, may be maintained near or adjacent to the proximal end 216 of the connector block 205. In other instances, the collet assembly 210 may be positioned closer the distal end 218 of the connector block 205 than the proximal end 216 of the connector block 205.

The spring 212 may be configured to bias the collet assembly 210 toward the proximal end 216 of the connector block 205. The spring 212 may maintain a position of the collet assembly 210 and oppose the insertion force of the portion of the implantable lead into the collet assembly 210. The spring 212 may force the collet assembly 210, and more particularly the one or more bearings 232 of the collet assembly 210, to engage with the angled inner circumferential surface 260 of the connector block 205 such that the collet assembly 210 collapses against and secures the portion of the implantable lead within the collet assembly 210.

The spring 212 may maintain engagement between the one or more bearings 232 of the collet assembly 210 and the angled inner circumferential surface 260 of the connector block 205 to releasably secure the portion of the implantable lead within the collet assembly 210. The portion of the implantable lead may be released by moving the collet assembly 210 toward the distal end 218 of the connector block 205 (e.g., by using a tool as described further hereinafter). The spring 212 may only compress as much as required to maintain at least a portion of the collet assembly 210 proximal to the proximal end 216 of the connector block 205 during insertion. As such, when force is removed from the implantable lead after insertion, the spring 212 may again cause the one or more bearings 232 of the collet assembly 210 to engage with the angled inner circumferential surface 260 of the connector block 205.

As noted above, the collet assembly 210 may be configured to remove pressure from the implantable lead in response to a tool moving the collet assembly 210 toward the distal end 218 of the connector block 205. As the tool moves the collet assembly 210 in this way, the collet assembly 210 may open or release forces on the portion of the implantable lead to allow the portion of the implantable lead to be removed. The tool may be an external tool (e.g., a probe) or an internal tool (e.g., an actuator such as a button with a mounted probe or molded piece) and may be configured to removable engage the collet assembly 210 (e.g., at a tip of the probe) through the access hole 224. For example, as discussed further hereinafter, the probe may have a profiled tip (e.g., chamfered or rounded) configured to engage a portion of the collet assembly 210.

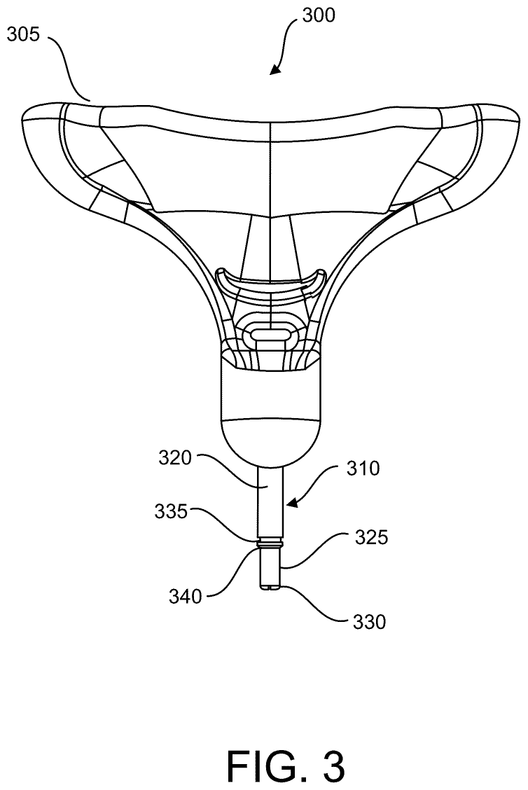

FIG. 3 is an elevation view of an exemplary release tool 300 for use with the connector port assembly 200, in accordance with embodiments of the present disclosure. As shown, in the illustrated embodiment, the release tool 300 includes a handle portion 305 and a shaft 310. As further shown, the shaft 310 has a proximal portion 320 and a distal portion 325 opposite the proximal portion 320, the distal portion 325 terminating at a distal end 330. In the illustrated embodiment, the shaft 310 also includes a circumferential notch 335 located at an intersection between the proximal portion 320 and the distal portion 325. In the particular embodiment illustrated in FIG. 3, the proximal portion 320 is larger in diameter than the distal portion 325, and the circumferential notch 335 operates, in part, to engage the tool retainer feature 215 (e.g., by a releasable snap-fit engagement) to releasably secure the release tool 300 to the connector port assembly 200 for removal of a lead terminal pin inserted therein. Additionally, in the illustrated embodiment, a shoulder 340 is defined at the transition between the proximal portion 320 and the distal portion 325, which is located so as to abut a corresponding surface of the connector 142 (FIG. 1) to limit the insertion depth of the shaft 310 of the release tool 300. Additionally, the distal end 330 of the shaft 310 has a contoured or chamfered outer periphery to facilitate smooth interaction with the collet body 230 (FIGS. 2A-2E) of the connector port assembly 200.

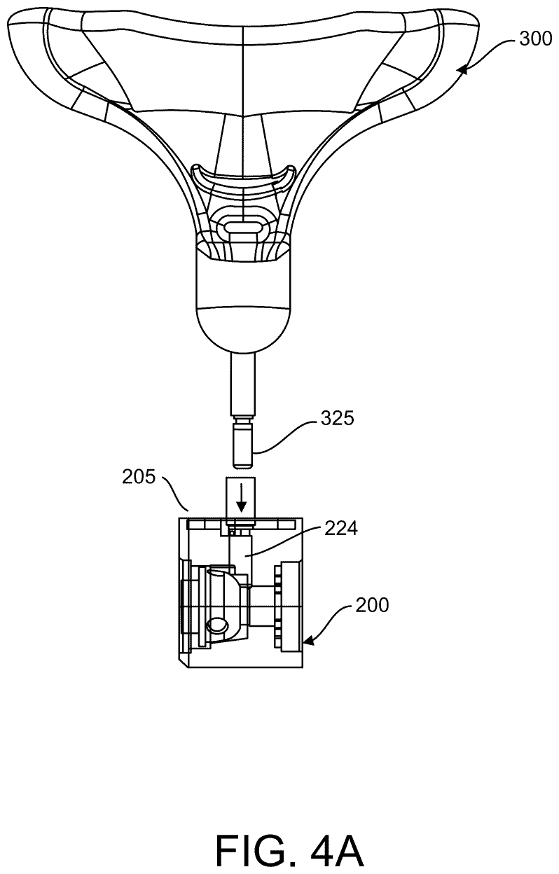

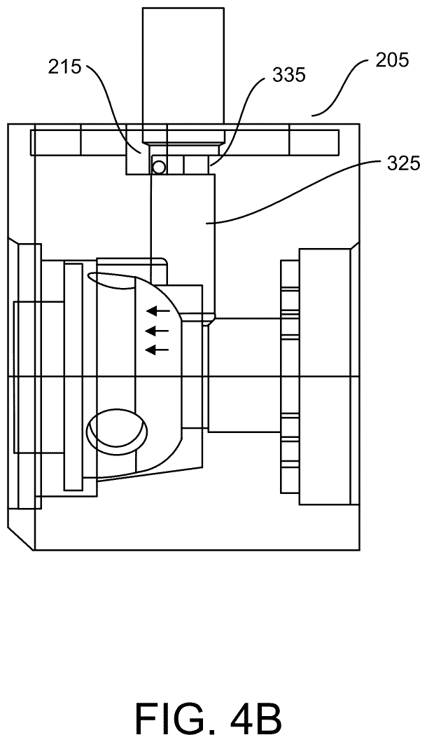

FIGS. 4A and 4B illustrate the release tool 300 in use in conjunction with the connector port assembly 200, in accordance with embodiments of the present disclosure. Referring to FIGS. 3 and 4A-4B together, the distal portion 325 of the release tool shaft 310 is dimensioned to be received within the access hole 224 of the connector block 205. As shown in FIG. 4B, when the distal portion 325 of the release tool shaft 310 is fully inserted into the access hole 224 of the connector block 205, the distal end 330 (FIG. 3) of the release tool shaft 310 engages the forward end portion 236 of the collet body 230 (FIGS. 2C, 2D) to urge the collet body 230 distally within the connector block 205 to release the compressive load on the lead terminal pin as described above. As previously described, the curved profile of the forward end portion 236 of the collet body 230 enables smooth translational movement of the collet body 230 when the release tool shaft 310 is pushed into the access hole 224, reducing the likelihood that the engaging surfaces will stick or that the distal end 330 of the release tool shaft 310 will gouge or otherwise damage the collet body 230. As further illustrated, when the release tool shaft 310 is fully inserted into the access hole 224, the circumferential notch 335 receives the release tool retainer 215 to releasably retain the release tool shaft 310 in the access hole 224, obviating the need for the user involvement to hold the release tool 300 in place during removal of the lead from the connector block. Additionally, when the lead removal is completed, the resiliency in the release tool retainer 215 (as discussed above) enables the user to remove the release tool shaft 310 from the access hole 224.

In various embodiments, the release tool shaft 310 and the access hole 224 are dimensioned and otherwise configured to limit the insertion length of the release tool shaft 310 into the access hole 224, e.g., to avoid placing excessive forces on the collet body 230 during lead removal. For example, in embodiments, the shoulder 340 on the release tool shaft 310 may be located and configured to abut a corresponding surface of the upper portion 264 of the access hole 224 to provide an insertion stop, with the length of the distal portion 325 of the release tool shaft 310 extending below the shoulder 340 dimensioned to provide a desired degree of engagement between the distal end 330 of the release tool shaft 310 and the connector body 230 to accomplish the aforementioned axial movement of the connector body 230 and allow for removal of the lead. In some embodiments, the distal portion 320 of the release tool shaft 310 and the corresponding lower portion 266 of the access hole 224 may have a frusto-conical profile and be dimensioned to provide the desired limits on insertion depth.

In some embodiments, the releasable retention of the release tool shaft 310 within the access hole 224 may be provided by additional or alternative means, i.e., either in addition to or in lieu of the inclusion of the release tool retainer 215. For example, in embodiments, the distal portion 325 of the release tool shaft 310 and the lower portion 266 of the access hole 224 may be dimensioned to provide a slight interference fit therebetween, sufficient to hold the distal portion 325 within the lower portion 266 while at the same time allowing for insertion of the distal portion 325 into the lower portion 266 and subsequent removal therefrom without requiring excessive force. In embodiments, cross-sectional profiles of the distal portion 325 of the release tool shaft 310 and the lower portion 266 of the access hole 224 may be different and have different dimensions to provide for the foregoing interference fit. For example, in embodiments, the lower portion 266 may have a circular cross-section and the distal portion 325 may have an elliptical cross-section with a major dimension slightly larger than the diameter of the lower portion 266 to provide the interference fit. In still other embodiments, the distal portion 325 of the release tool shaft 310 may be bent slightly with sufficient flexibility to permit insertion into the access hole 224, e.g., to operate similarly to a leaf spring.

Aspects of the present disclosure, including those discussed in relation to the figures, provide numerous advantages. For example, typical applications of a connector port employ a combination of a tip block, a set screw, and a torque wrench to secure and release an implantable lead. By removing these components, the present disclosure reduces the amount of noise issues in the field from no or under torqued set screws. In addition, usability is improved because the present disclosure allows for easy attachment of the implantable lead and because relative axial motion of the lead within the connector port may be inhibited. As well, the overall design of the IMD may be improved because the need for certain components (e.g., top mounted seal plugs) may be eliminated. By simplifying the design of the retaining mechanism and eliminating certain components, the profile of the header may also be improved. Plus, the associated manufacturing process may be simple.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An implantable medical device comprising:
a housing;
a header arranged with the housing; and
a connector port assembly arranged within the header and configured to couple an implantable lead to the header, the connector port assembly including:
a connector block having an upper surface, a proximal end, an opposite distal end, a connector block bore extending between the proximal and distal ends, and an access hole extending radially from the upper surface to the connector block bore between the proximal and distal ends of the connector block, the access hole oriented generally orthogonally to an axis of the connector block bore; and
a collet assembly including a collet body and a plurality of bearings arranged about a circumference of the collet body, the plurality of bearings configured to contact an inner surface of the connector block bore and to frictionally engage a portion of the implantable lead to secure the implantable lead with the header in response to insertion of the portion of the implantable lead into the connector block bore, the collet body including a forward end portion having a curved outer surface,
wherein the access hole of the connector block is aligned with curved outer surface of the forward end portion of the collet body.

2. The implantable medical device of claim 1, wherein the plurality of bearings translate toward the distal end of the connector block in response to insertion of the portion of the implantable lead into the connector block bore.

3. The implantable medical device of claim 2, further comprising a spring element disposed within the connector block bore and about the distal end portion of the collet body, the spring element configured to bias the collet body toward the proximal end of the connector port.

4. The implantable medical device of claim 3, wherein the spring element has a serpentine shape when viewed in a direction orthogonal to a longitudinal axis of the connector port.

5. The implantable medical device of claim 4, wherein the distal portion of the collet body is dimensioned to maintain axial alignment of the collet body during axial translation of the collet body.

6. An implantable medical device comprising:
a housing;
a header arranged with the housing; and
a connector block arranged within the header and having an upper surface, a proximal end, an opposite distal end, a connector block bore extending between the proximal and distal ends, and an access hole extending radially through the connector block from the upper surface to the connector block bore and between the proximal and distal ends of the connector block, the access hole being oriented orthogonally to an axis of the connector block bore and sized to receive a shaft of a release tool, the connector block further including a release tool retaining feature; and
a collet assembly including a collet body and a plurality of bearings arranged about a circumference of the collet body, the plurality of bearings configured to contact an inner surface of the connector block bore and to frictionally engage a portion of an implantable lead to secure the implantable lead with the header in response to insertion of the portion of the implantable lead into the connector block bore, the collet body including a forward end portion and a distal portion opposite the forward end portion,
wherein the access hole of the connector port is positioned such that a shaft of the release tool can engage the forward end portion of the collet body, and the release tool retaining feature is configured to frictionally retain the release tool within the access hole in engagement with the forward end portion of the collet body.

7. The implantable medical device of claim 6, wherein the tool retaining feature is a resilient member extending across the access hole.

8. The implantable medical device of claim 7, wherein the resilient member includes opposite end portions that are each retained within a slot formed in the connector block adjacent to the access hole, and a center portion that spans across the access hole.

9. The implantable medical device of claim 8, wherein the plurality of bearings translate toward the distal end of the connector block in response to insertion of the portion of the implantable lead into the connector block bore.

10. The implantable medical device of claim 9, further comprising a spring element disposed within the connector block bore and about the distal end portion of the collet body, the spring element configured to bias the collet body toward the proximal end of the connector block.

11. The implantable medical device of claim 10, wherein the spring element has a serpentine shape when viewed in a direction orthogonal to a longitudinal axis of the connector port.

12. The implantable medical device of claim 11, wherein the distal portion of the collet body is dimensioned to maintain axial alignment of the collet body during axial translation of the collet body.

13. A connector port assembly for releasably securing an implantable lead to a header of an implantable medical device, the connector port assembly comprising:
a connector block having an upper surface, a proximal end, an opposite distal end, a connector block bore extending between the proximal and distal ends, and an access hole extending radially through the connector block from the upper surface to the connector block bore and between the proximal and distal ends of the connector block, the access hole being oriented orthogonally to an axis of the connector block bore and sized to receive a shaft of a release tool; and
a collet assembly including a collet body and a plurality of bearings arranged about a circumference of the collet body, the plurality of bearings configured to contact an inner surface of the connector block bore and to frictionally engage a portion of an implantable lead to secure the implantable lead with the connector port assembly in response to insertion of the portion of the implantable lead into the connector block bore, the collet body including a forward end portion having a curved outer surface, and a distal portion opposite the forward end portion,
wherein the access hole of the connector block is positioned such that a shaft of the release tool can engage the curved outer surface of the forward end portion of the collet body.

14. The connector port assembly of claim 13, wherein the connector block further comprises a release tool retaining feature configured to frictionally retain the release tool within the access hole in engagement with the curved outer surface of the forward end portion of the collet body.

15. The connector port assembly of claim 14, wherein the release tool retaining feature is a resilient member extending across the access hole.

16. The connector port assembly of claim 15, wherein the resilient member includes opposite end portions that are each retained within a slot formed in the connector block adjacent to the access hole, and a center portion that spans across the access hole.

17. The connector port assembly of claim 16, wherein the plurality of bearings translate toward the distal end of the connector block in response to insertion of the portion of the implantable lead into the connector block bore.

18. The connector port assembly of claim 14, further comprising a spring element disposed within the connector block bore and about the distal end portion of the collet body, the spring element configured to bias the collet body toward the proximal end of the connector block.

19. The implantable medical device of claim 18, wherein the spring element has a serpentine shape when viewed in a direction orthogonal to a longitudinal axis of the connector port.

20. The connector port assembly of claim 14, wherein the distal portion of the collet body is dimensioned to maintain axial alignment of the collet body during axial translation of the collet body.
